(19) United States
(12) Patent Application Publication
(10) Pub. No.: US 2026/0226355 A1
(43) Pub. Date: Aug. 6, 2026

(54) METHOD FOR MONITORING AND CONTROLLING SALT CONTENT OF DESALTED CRUDE OIL IN GAS-OIL SEPARATION PROCESS

(71) Applicant: SAUDI ARABIAN OIL COMPANY, Dhahran (SA)

(72) Inventors: Ammar Mustafa Alsaeed, Dammam (SA); Nisar Ahmad K. Ansari, Ras Tanura (SA)

(73) Assignee: SAUDI ARABIAN OIL COMPANY, Dhahran (SA)

(21) Appl. No.: 19/042,430

(22) Filed: Jan. 31, 2025

(51) Int. Cl. C10G 29/02; G01N 33/28

(52) U.S. Cl. CPC C10G 29/02; G01N 33/2835; C10G 2300/1037

(57) ABSTRACT

A method for monitoring and controlling a salt content of desalted crude oil includes separating a dehydrator feed stream into a crude and water effluent streams, feeding the crude effluent stream from the dehydrator and a wash water stream to a desalter, separating the mixture into a desalter crude and water effluent streams, and recirculating at least a portion of the desalter water effluent stream to the dehydrator. The method includes measuring flowrates of the wet crude feed, wash water, and water recirculation streams, a water content of the wet crude feed stream, a salt content of the wet crude feed and wash water stream. The method includes iteratively calculating a salt content of the desalter crude effluent stream according to salt mass balances around the dehydrator and the desalter, based on target BS&W of the dehydrator and desalter crude effluent streams.

### BACKGROUND

In Gas-Oil Separation Plants (GOSPs), multiphase feed crude is separated into crude oil, produced water and gases. The GOSPs may include a Wet Crude Handling (WCH) system which includes crude dehydration and crude desalting processes. These processes help remove water and dissolved salts from the feed crude. Removal of these components is required to prevent corrosion and fouling/poisoning of catalysts in the downstream refinery processes. In order to ensure proper removal of salts from the crude oil, maintain the desalted crude oil quality, and help optimize the process efficiency, monitoring the salt content in the produced crude oil which has undergone dehydration and desalting process is essential.

Monitoring of the salt content in crude oil often involves dedicated salt analyzers, which can be costly to install and maintain. Furthermore, the salt analyzers require periodic calibration, maintenance, and troubleshooting and repair when not functioning properly, which may lead to increased downtime of the dehydration and desalting operation. The salt analyzers may also require handling of crude oil samples and hazardous chemical agents, which may pose safety and environmental concerns. Accordingly, there exists a need for improved monitoring and controlling of the desalted crude oil salt content in GOSPs.

### SUMMARY

This summary is provided to introduce a selection of concepts that are further described below in the detailed description. This summary is not intended to identify key or essential features of the claimed subject matter, nor is it intended to be used as an aid in limiting the scope of the claimed subject matter.

In one aspect, embodiments disclosed herein relate to a method for monitoring a salt content of desalted crude oil. The method includes feeding a dehydrator feed stream including a wet crude feed stream and a water recirculation stream to a dehydrator, separating the dehydrator feed stream into a dehydrator crude effluent stream and a dehydrator water effluent stream, feeding a desalter feed stream including the dehydrator crude effluent stream and a wash water stream to a desalter unit, mixing the desalter feed stream in the desalter unit, separating the desalter feed stream into a desalter crude effluent stream and a desalter water effluent stream, and recirculating at least a portion of the desalter water effluent stream to the dehydrator as the water recirculation stream.

The method further includes measuring flowrates of the wet crude feed stream, wash water stream, and water recirculation stream, measuring a water content of the wet crude feed stream, and measuring a salt content of the wet crude feed stream and the wash water stream.

The method also includes obtaining a target basic sediment and water (BS&W) of the dehydrator crude effluent stream and the desalter crude effluent stream, and iteratively calculating a salt content of the desalter crude effluent stream according to salt mass balances around the dehydrator and the desalter, based on input parameters and a trial salt content value of the desalter crude effluent stream. The input parameters include the flowrates of the wet crude feed stream, the wash water stream, and the water recirculation stream, the water content of the wet crude feed stream, the salt content of the wet crude feed stream and the wash water stream, and the target BS&W of the dehydrator crude effluent stream and the desalter crude effluent stream.

In another aspect, embodiments herein relate to a method for controlling a salt content of desalted crude oil. The method includes feeding a dehydrator feed stream including a wet crude stream and a first water recirculation stream to a dehydrator, separating the dehydrator feed stream into a dehydrator crude effluent stream and a dehydrator water effluent stream, feeding a first desalter feed stream including the dehydrator crude effluent stream and a second water recirculation stream to a first desalter unit, mixing the first desalter feed stream in the first desalter unit, separating the first desalter feed stream into a first desalter crude effluent stream and a first desalter water effluent stream, and recirculating at least a portion of the first desalter water effluent stream to the dehydrator as the first water recirculation stream.

The method further includes feeding a second desalter feed stream including the first desalter crude effluent stream and a wash water stream to a second desalter unit, mixing the second desalter feed stream in the second desalter unit, separating the second desalter feed stream into a second desalter crude effluent stream and a second desalter water effluent stream, and recirculating at least a portion of the second desalter water effluent stream to the first desalter unit as the second water recirculation stream.

The method also includes periodically measuring flowrates of the wet crude feed stream, the wash water stream, the first water recirculation stream, and the second water recirculation stream, a water content of the wet crude feed stream, and a salt content of the wet crude feed stream and the wash water stream, obtaining target parameters including a target basic sediment and water (BS&W) of the dehydrator crude effluent stream, the first desalter crude effluent stream and the second desalter crude effluent stream, and iteratively calculating a salt content of the second desalter crude effluent stream according to salt mass balances around the dehydrator, the first desalter and the second desalter, based on input process parameters, the target parameters and a trial salt content value of the second desalter crude effluent stream.

The method further includes comparing the salt content of the second desalter crude effluent stream and a target salt content of the second desalter crude effluent stream, and when the salt content of the second desalter crude effluent is equal or greater than the target salt content of the second desalter crude effluent stream, adjusting at least one of the input process parameter or the target parameter; and repeating the iteratively calculating, the comparing, and the adjusting steps until the salt content of the second desalter crude effluent stream is less than the target salt content. The input process parameters include flowrates of the wet crude feed stream, wash water stream, and first and second water recirculation streams, the water content of the wet crude feed stream, and the salt content of the wet crude feed stream and the wash water stream.

Other aspects and advantages of the claimed subject matter will be apparent from the following description and the appended claims.

### BRIEF DESCRIPTION OF DRAWINGS

FIG. 1 is a schematic diagram of a continuous crude oil desalting system according to one or more embodiments.

FIG. 2 is a schematic diagram of a continuous crude oil desalting system according to one or more embodiments.

FIGS. 3A-3B illustrate input parameters and calculated results for the method of EXAMPLE 1 according to one or more embodiments.

FIG. 4 is a graph of calculated salt content in EXAMPLE 1 according to one or more embodiments, and salt contents of the desalted crude oil obtained via lab analysis.

### DETAILED DESCRIPTION

In one aspect, embodiments disclosed herein relate to a method for monitoring a salt content of desalted crude oil. The method includes, in a continuous crude oil desalting system, obtaining or measuring input parameters, and iteratively calculating a salt content of the desalter crude effluent stream according to salt mass balances. The method is conducted to monitor the salt content of desalted crude oil without the use of a salt analyzer.

The method for monitoring the salt content may be a continuous monitoring of the salt content of desalted crude oil. In the present disclosure, a “continuous monitoring” refers to monitoring based on discrete but substantially frequent data acquisition and salt content calculation, for example, based on digital data acquisition and calculation, such that the calculated salt content values and change in salt content values represent the actual salt content values and change is the salt content values.

### Continuous Crude Oil Desalting System

In one or more embodiments, the method for monitoring and controlling a salt content of desalted crude oil is conducted in a continuous crude oil desalting system. The continuous crude oil desalting system may include a dehydrator and a desalter fluidly connected to the dehydrator. The dehydrator may be configured to receive a dehydrator feed stream including a wet crude feed stream and a water recirculation stream, and separate the dehydrator feed stream into a dehydrator crude effluent stream and a dehydrator water effluent stream. The dehydrator may be configured to receive a desalter feed stream including the dehydrator crude effluent stream and a wash water stream, mix the desalter feed stream, and separate the desalter feed stream into a desalter crude effluent stream and a desalter water effluent stream.

In the present disclosure, a “wet crude” refers to a crude oil that includes water. The water included in the wet crude may be formation water which may include salt and/or other impurities. In the present disclosure, a “salt” refers to inorganic salt typically contained in the formation water included in the wet crude oil. The salt may include, but are not limited to, sodium chloride, magnesium chloride, calcium chloride, calcium sulfate, sodium carbonate and magnesium carbonate. A “salt content” refers to the amount of salt in a specific fluid stream, such as crude oil stream or water stream, and is expressed in terms of the mass of the salt per unit volume of the fluid, such as milligram of salt per liter of a fluid (mg/l). The fluid may be water, crude oil or combinations thereof.

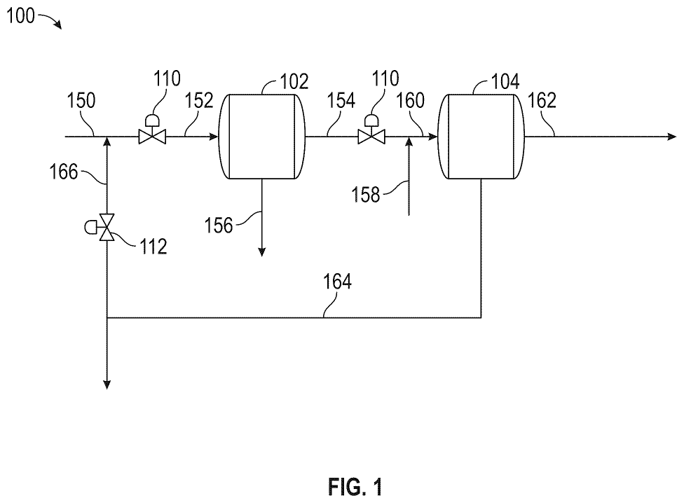

FIG. 1 is a schematic diagram of a continuous crude oil desalting system of one or more embodiments. The continuous crude oil desalting system (“desalting system”) 100 includes a dehydrator 102 and a desalter unit (“desalter”) 104 that is fluidly connected to the dehydrator 102. The dehydrator 102 may be a horizontal vessel where the first stage of drying the wet crude oil takes place. A dehydrator feed stream 152, which includes a wet crude feed stream 150 and a water recirculation stream 166 from the desalter 104, enters the dehydrator 102. The dehydrator 102 separates, via electrostatic coalescence, the dehydrator feed stream 152 into a dehydrator water effluent stream 156 and a dehydrator crude effluent stream 154. The dehydrator 102 may agitate and mix the dehydrator feed stream 152, which includes the wet crude feed stream 150 and the water recirculation stream 166 prior to the separation step. A portion of water included in the dehydrator feed stream 152 may remain in the dehydrator crude effluent stream 154. The amount of water in the dehydrator crude effluent stream 154 is specified based on the maximum allowable basic sediment and water (BS&W), which is described in the subsequent sections.

The dehydrator crude effluent stream 154 exits the dehydrator 102 and combines with a wash water stream 158 to become a desalter feed stream 160. The wash water dilutes the salt included in the water phase of the desalter feed stream 160. The desalter feed stream 160 enters the desalter 104 which agitates and mixes the desalter feed stream 160 and separates the desalter feed stream 160 into a desalter crude effluent stream 162 and a desalter water effluent stream 164. The desalter crude effluent stream 162 is then passed through a depressurization valve, retrieved, and may be processed further, for example, to adjust the crude vapor pressure by removing light hydrocarbons (such as C1-C4) (“stabilization” process), and reduce the H₂S content (“sweetening” of crude oil”) to meet the required values in a specification. At least a portion of the desalter water effluent stream 164 is recirculated back as a water recirculation stream 166 and combined with the wet crude feed stream 150 to enter the dehydrator 102. The dehydrator water effluent stream 156 and a portion of the desalter water effluent stream 164 that is not recirculated are retrieved and may undergo a purification process, such as a water and oil separation process.

The dehydrator 102 and the desalter 104 may be operated at a temperature in a range of from about 130 to 160° F., and at a pressure of about 25 psig above the crude vapor pressure.

The system 100 may include mixing valves 110 at the inlet of the dehydrator 102 and the desalter 104 to promote agitation and mixing of the feed substances, such as the wet crude feed stream, recirculation water streams and wash water streams, and efficient dehydration and desalting processes. The dehydrator 102 and the desalter 104 may be vessels including electrostatic grids inside of the vessels to generate electrostatic field in order to promote coalescence of small colloidal droplets present in the emulsion into larger droplets and separation of water from hydrocarbons.

The system 100 may include water recirculation stream valves 112 to control a percent recycling ratio of the desalter water effluent stream.

In one or more embodiments, the continuous crude oil desalting system includes a dehydrator, a first stage desalter (“first desalter”) fluidly connected to the dehydrator, and a second stage desalter (“second desalter”) fluidly connected to the first desalter. The dehydrator may be configured to receive a dehydrator feed stream including a wet crude feed stream and a first water recirculation stream, mix the dehydrator feed stream, and separate the dehydrator feed stream into a dehydrator crude effluent stream and a dehydrator water effluent stream. The first desalter may be configured to receive a first desalter feed stream including the dehydrator crude effluent stream and a second water recirculation stream, mix the first desalter feed stream, and separate the first desalter feed stream into a first desalter crude effluent stream and a first desalter water effluent stream. The second desalter may be configured to receive a second desalter feed stream including the first desalter crude effluent stream and a wash water stream, mix the second desalter feed stream, and separate the second desalter feed stream into a second desalter crude effluent stream and a second desalter water effluent stream.

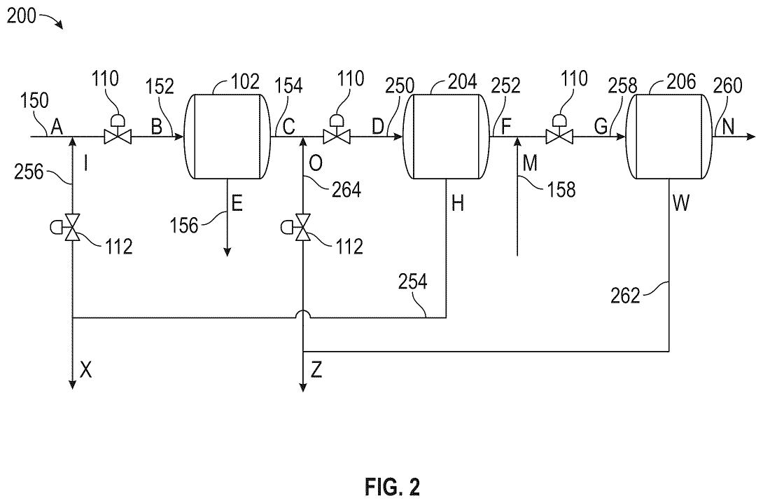

FIG. 2 a schematic diagram of a continuous crude oil desalting system of one or more embodiments. The continuous crude oil desalting system (“desalting system”) 200 includes a dehydrator 102, a first stage desalter (“first desalter,” “first desalter unit”) 204 that is fluidly connected to the dehydrator 102, and a second stage desalter (“second desalter,” “second desalter unit”) 206 that is fluidly connected to the first desalter 204. A dehydrator feed stream 152, which includes a wet crude feed stream 150 and a first water recirculation stream 256 from the first desalter 204, enters the dehydrator 102. The dehydrator 102 separates the dehydrator feed stream 152 into a dehydrator water effluent stream 156 and a dehydrator crude effluent stream 154. A portion of water included in the dehydrator feed stream 152 may remain in the dehydrator crude effluent stream 154.

The dehydrator crude effluent stream 154 exits the dehydrator 102 and combines with a second water recirculation stream 264 from the second desalter 206 to become a first desalter feed stream 250. The second water recirculation stream 264 dilutes the salt included in the water phase of the first desalter feed stream 250. The first desalter feed stream 250 enters the first desalter 204 which agitates and mixes the first desalter feed stream 250 and separates the first desalter feed stream 250 into a first desalter crude effluent stream 252 and a first desalter water effluent stream 254.

The first desalter water effluent stream 254 exits the first desalter 204. At least a portion of the first desalter water effluent stream 254 is recirculated back as the first water recirculation stream 256 and combined with the wet crude feed stream 150 to enter the dehydrator 102 as the dehydrator feed stream 152.

The first desalter crude effluent stream 252 exits the first desalter 204 and combines with a wash water stream 158 to become a second desalter feed stream 258. The wash water stream 158 further dilutes the salt included in the water phase of the second desalter feed stream 258. The second desalter feed stream enters the second desalter 206 which agitates and mixes the second desalter feed stream 258 and separates the second desalter feed stream 258 into a second desalter crude effluent stream 260 and a second desalter water effluent stream 262.

The second desalter water effluent stream 262 exits the second desalter 206. At least a portion of the second desalter water effluent stream 262 is recirculated back as the second water recirculation stream 264 and combined with the dehydrator crude effluent stream 154 to enter the first desalter 204 as the first desalter feed stream 250.

The second desalter crude effluent stream 260 exits the second desalter 206 and retrieved to be processed further to adjust, for example, the crude vapor pressure and the H₂S content to meet the specification values.

The dehydrator water effluent stream 156 and a portion of the first and second desalter water effluent stream that is not recirculated are retrieved and may undergo a purification process, such as a water and oil separation process.

The desalting system 200 may include mixing valves 110 at the inlet of the dehydrator 102, the first desalter 204 and the second desalter 206. The desalting system 200 may include water recirculation stream valves 112 to control a percent recycling ratio of the first desalter water effluent stream 254 and the second desalter water effluent stream 262.

The embodiments in FIG. 1 and FIG. 2 include one dehydrator and one or two desalters. However, the present disclosure is not limited to the embodiments of FIGS. 1 and 2, and may include a plurality of dehydrators and/or 3 or more desalters in various configurations, such as in series or in parallel, based on the requirements of a specific application.

In one or more embodiments, the continuous crude oil desalting system additionally includes at least one of production separators, low pressure production trap (LPPT), heat exchangers, low pressure degassing tanks (LPDT), and a trim heater, upstream of the dehydrator. The continuous crude oil desalting system may include a water/oil separation plant (WOSEP) where the non-recirculated portion of the water effluent streams from the dehydrator and desalter is introduced and the residual crude oil is separated from water. The separated water may be discharged to a disposal water well, and the residual crude oil may be introduced to LPDT. The continuous crude oil desalting system may further include pumps, reboilers and a stabilizer columns in appropriate locations a based on the design requirements of each configuration.

### Wet Crude Feed Stream

In one or more embodiments, the wet crude feed stream includes hydrocarbons, water and salt. The wet crude feed stream may include water in an amount of from about 1 vol % to about 40 vol %, based on the total amount of the wet crude feed stream. The wet crude feed stream may include water in an amount of from a lower limit selected from any one of 1, 2, and 5 vol % to an upper limit selected from any one of 10, 11, 15, 20, 30 and 40 vol %, where any lower limit may be paired with any upper limit.

In one or more embodiments, the wet crude feed stream includes a salt in an amount of from about 50,000 mg/l to about 250,000 mg/l, such as in a range of from a lower limit selected from any one of 50,000, 75,000, and 100,000 mg/l to an upper limit selected from any one of 150,000, 200,000, and 250,000 mg/l, where any lower limit may be paired with any upper limit.

Prior to being introduced to the continuous crude desalting system 100 or 200, the wet crude feed stream may undergo various separation processes to remove most of the gasses and free formation water. Such separation processes may include, but are not limited to, production separators, LPPT, a heat exchanger and LPDT. The wed crude may initially undergo a three-phase separation to remove most of the gasses and free-formation water. The pressure drop in the production separator causes the lighter hydrocarbon gases in the crude oil to separate from the heavier liquid hydrocarbons. The water discharge stream is discharged for collection to the water/oil separator vessel. The operating conditions in the production separator ranges from 65° F. to 130° F. and about 30 to 150 psig.

In LPPT, the wet crude is subjected to a temperature in a range of from about 65 to 130° F. and a pressure of about 30 to 150 psig to separate hydrocarbon gases from heavier liquid hydrocarbon due to reduced pressure. The wet crude oil stream from LPPT is then passed through a heat exchanger to reheat the oil/water/gas mixture, which allows easier water separation from the crude oil. The oil/water/gas mixture from the heat exchanger is then fed through the LPDT where the pressure is reduced to 3 psig, for example, such that the heavier gas components can be removed from the mixture. The operating temperature and pressure of the LPDT may be in a range of from about 65 to 130° F. and 3 to 5 psig, respectively. The wet crude from LPDT may be pumped through crude charge pumps and introduced to a trim heater to increase the temperature prior to being introduced to the dehydrator 102 or 202 of the continuous crude desalting system 100 or 200.

In one or more embodiments, the wet crude feed stream includes demulsifying agent, which is added in the upstream process, in order to promote separation of emulsified water in the wet crude feed stream. The addition of the demulsifying agent may be conducted during the heating operation of the wet crude oil.

In one or more embodiments, the wet crude feed stream may have a flowrate in a range of from about 100 million barrels per day (MBD) to about 900 MBD, such as in a range of from a lower limit selected from any one of 100, 200, 300 and 400 MBD to an upper limit selected from any one of 600, 700, 800 and 900 MBD, where any lower limit may be paired with any upper limit.

### Wash Water Stream

In one or more embodiments, the wash water stream is free of total dissolved solids (TDS), such as salts, or has substantially low TDS. For example, the wash water stream may include a salt in an amount of from about 200 mg/l to about 10000 mg/l, such as in a range of from a lower limit selected from any one of 200, 500 and 1000 mg/l to an upper limit selected from any one of 300, 500, 1000, 5000 and 10000 mg/l, where any lower limit may be paired with any mathematically-compatible upper limit.

In one or more embodiments, the wash water stream has a flowrate in a range of from about 0.5 vol % to 5 vol % of the flowrate of a dry crude feed stream, which is a hydrocarbon portion of the wet crude feed stream, or a portion of the wed crude feed stream excluding the water. The wash water stream may have a flowrate in a range of from a lower limit selected from any one of 0.5, 0.75 and 1 vol %, to an upper limit selected from any one of 2, 3, 4 and 5 vol %, of the flowrate of the dry crude feed stream, where any lower limit may be paired with any upper limit.

### Desalted Crude Oil

In one or more embodiments, the desalted crude oil (corresponding to the desalter crude effluent stream in the embodiments of FIG. 1 and the second desalter crude effluent stream in the embodiments of FIG. 2) may have a salt content of equal or less than about 10 lb of salt per 1000 barrels of crude (PTB) such as less than 10, 8, 6, 5, or 4 PTB, or in a range of from a lower limit selected from any one of 0, 0.1 and 0.5 PTB, to an upper limit selected from any one of 4, 5, 7, 8, and 10 PTB, where any lower limit may be paired with any upper limit. The salt content of the desalted crude oil may be determined based on a required value of a specification.

The desalted crude oil may have a basic sediment and water (BS&W) value of less than about 0.3 vol % based on the total volume of the desalted crude oil, such as less than 0.3, equal or less than 0.2 or equal or less than 0.1 vol %, A BS&W refers to the amount of impurities contained in the crude oil and may include water and suspended solids.

The desalted crude oil may have a hydrogen sulfide (H₂S) content of less than 60 ppm, such as in a range of from about 10 ppm to 60 ppm. In one or more embodiments, the desalted crude oil may be further processed (sweetening process), for example, in a crude stabilization tower or a degassing vessel, to have a H₂S of less than 60 ppm, such as in a range of from about 10 ppm to 60 ppm.

The desalted crude oil may have a maximum Reid vapor pressure (RVP) of 7 psia and a maximum true vapor pressure (TVP) of 13.5 psia or 13 psia at 130° F. The desalted crude oil having the RVP and TVP in the aforementioned range does not provide sufficient vapor under a standard atmospheric pressure of, for example, 14.7 psia, to flash. Such desalted crude oil can be transported safety.

### A Method for Monitoring a Salt Content of Desalted Crude Oil

In one or more embodiments, the method for monitoring a salt content of desalted crude oil includes feeding a dehydrator feed stream including a wet crude feed stream and a water recirculation stream to a dehydrator and separating the dehydrator feed stream into a dehydrator crude effluent stream and a dehydrator water effluent stream.

The method may further include feeding a desalter feed stream including the dehydrator crude effluent stream and a wash water stream to a desalter unit, mixing the desalter feed stream in the desalter unit, separating the desalter feed stream into a desalter crude effluent stream and a desalter water effluent stream, and recirculating at least a portion of the desalter water effluent stream to the dehydrator as the water recirculation stream. The method may be conducted, for example, in a continuous crude oil desalting system as described in FIG. 1.

In one or more embodiments which includes first and second desalters, the method includes feeding a dehydrator feed stream including a wet crude stream and a first water recirculation stream to a dehydrator, and separating the dehydrator feed stream into a dehydrator crude effluent stream and a dehydrator water effluent stream.

The method may further include feeding a first desalter feed stream including the dehydrator crude effluent stream and a second water recirculation stream to a first desalter unit, mixing the first desalter feed stream in the first desalter unit, separating the first desalter feed stream into a first desalter crude effluent stream and a first desalter water effluent stream, and recirculating at least a portion of the first desalter water effluent stream to the dehydrator as the first water recirculation stream.

The method may also include feeding a second desalter feed stream including the first desalter crude effluent stream and a wash water stream to a second desalter unit, mixing the second desalter feed stream in the second desalter unit, separating the second desalter feed stream into a second desalter crude effluent stream and a second desalter water effluent stream, and recirculating at least a portion of the second desalter water effluent stream to the first desalter unit as the second water recirculation stream. The method may be conducted, for example, in a continuous crude oil desalting system as described in FIG. 2.

In one or more embodiments, the method includes obtaining or measuring input parameters. The input parameters may include input process parameters and target parameters. The input process parameters generally refer to parameters related to processing and properties of the crude oil and water stream. The input process parameters may include, but are not limited to, flow rates of the wet crude feed stream, the wash water stream, the water recirculation streams such as the first water recirculation stream, and the second water recirculation stream, a water content of the wet crude feed stream, and a salt content of the wet crude feed stream and the wash water stream. The input process parameters may be measured directly from various monitoring devices included in the system, such as a flow meter, or may be measured by testing a sample from the streams. In one or more embodiments, the water and salt content of the wet crude feed stream and the salt content of the wash water stream may remain unchanged for an extended period of time, such as five years.

The target parameters refer to parameters for which a specific value is selected for the purpose of determining the salt content of desalted crude oil in a specific instance. The target parameters may be selected based on various specifications, or may be predetermined based on specific process configurations.

The target parameters may include, but are not limited to, a target BS&W value of the dehydrator crude effluent stream, and a target BS&W value of the desalter crude effluent stream such as the first desalter crude effluent stream and the second desalter crude effluent stream. The target BS&W may be selected based on a required product specification, and may be, for example, in a range of from about 0 vol % to about 0.2 vol %. The target BS&W may be 0.2 vol %.

The target parameters may also include a mixing efficiency of the dehydrator, and the desalters such as the first desalter and the second desalter, and precent recycling ratio of the water recirculation stream, such as the first water recirculation stream and the second water recirculation stream, with respect to the water effluent stream from the desalter including the first desalter and the second desalter.

In one or more embodiments, the mixing efficiency of the dehydrator and desalters may be in a range of from about 50% to about 90%, such as in a range of from a lower limit selected from any one of 50%, 60% and 70%, to an upper limit selected from any one of 60%, 70%, 75%, 80%, and 90%, where any lower limit may be paired with any mathematically-compatible upper limit. The mixing efficiency of the dehydrator and desalters may be about 50%. The mixing efficiency may be influenced by the properties of the mixing valve of the dehydrator and desalters, such as valve age, valve effective controllability, which is used to control the blending of the feed streams including the dehydrator feed stream and the desalter feed stream. The mixing valve is used to achieve the required composition and uniformity of the composition.

In one or more embodiments, the target parameters include a percent recycling ratio of the water recirculation streams, such as the first water recirculation stream and the second water recirculation stream. The precent recycling ratio of the water recirculation stream may be in a range of from about 0.1% to about 100%, such as in a range of from a lower limit selected from any one of 0.1, 0.5, 1, 10, 20, 30 and 40%, to an upper limit selected from any one of 60%, 70%, 80%, 90%, and 100%, where any lower limit may be paired with any upper limit, based on the flow rate of the water effluent stream from the dehydrator or desalter. The percent recycling ration of the water recirculation stream may be 100%, which indicates that the entirety of the water effluent stream is recirculated. Based on the embodiments as shown in FIG. 2, the percent recycling ratio of the first water recirculation stream indicates a ratio of the flow rate of the first water recirculation stream and the flow rate of the first desalter water effluent stream, and the percent recycling ratio of the second water recirculation stream indicates a ratio of the flowrate of the second water recirculation stream and the flow rate of the second desalter water effluent stream.

In one or more embodiments, the method includes iteratively calculating a salt content of the desalter crude effluent stream according to salt mass balances around the dehydrator and the desalter, such as the first desalter and the second desalter, based on the input parameters and a trial salt content value of the desalter crude effluent stream.

In the present disclosure, iteratively calculating the salt content of the desalter crude effluent “according to salt balances” refers to calculating the salt content by conducting a salt mass balance around each of the apparatus included in the continuous crude oil desalting system, including the dehydrator and the desalter such as the first desalter and the second desalter. A salt balance is conducted by equating the amount of salt entering a specific apparatus to the amount of salt exiting the apparatus, assuming that there is no generation or consumption of the salt in the apparatus.

As an example, a salt mass balance around the dehydrator may be expressed as follows:

m_{sci}=m_{swo}+m_{sco}

where m_{sci} is a mass flow rate of salt in the dehydrator feed stream, m_{swo} is a mass flow rate of salt in the dehydrator water effluent stream and m_{sco} is a mass flow rate of salt in the dehydrator crude effluent stream. Each of m_{sci}, m_{swo} and m_{sco} may be determined by multiplying the flowrate and the salt content (salt concentration) of each streams.

A similar salt mass balance around the desalter, such as the first desalter and the second desalter can be obtained. The mass balance around the desalter may be expressed by equating a salt mass flowrate of the desalter feed stream, such as the first desalter feed stream, with the sum of a salt mass flowrate of the desalter water effluent stream, such as the first desalter water effluent stream and the second desalter water effluent stream, and the desalter crude effluent stream, such as the first desalter crude effluent stream and the second desalter crude effluent stream.

In the “iterative calculation” of the present disclosure, equations associated with the mass balance of each of the dehydrator and desalter as described above are simultaneously solved (initial trial step) based on the input parameters as previously described and a selected “initial” value of salt content the desalter crude effluent stream. The initial trial step then generates calculated values of salt contents of the dehydrator crude effluent stream, the dehydrator water effluent stream, the first crude effluent stream, the first desalter water effluent stream, the second desalter crude effluent stream, and the second desalter water effluent stream, which corresponds to the initial trial salt content of the desalted crude oil.

The equations are solved again based on a new, “subsequent” value of salt content of the desalter crude effluent stream, which differs from the initial salt content value by a predetermined step amount value (subsequent trial step) to obtain salt contents of the streams as described in the initial trial step. The predetermined step amount value maybe for example, 0.001% of the initial or subsequent value of the salt content of the desalter crude effluent stream. In one or more embodiments, the predetermined step amount value may be in a range of from greater than 0% to about 0.2%, such as in a range of from 0.00001% to 0.2% of the initial or subsequent value of the salt content of the desalter crude effluent stream.

A comparing step is then conducted by obtaining the differences of the salt contents of each stream from the initial trial step and the subsequent trail step are then obtained (delta value) and the delta value of each stream is compared to a predetermined convergence tolerance value. If the delta value of at least one of the stream is greater than the predetermined convergence tolerance value, the initial trial step, the subsequent trial step, and the comparing step are repeated by substituting the subsequent value of the salt content of the desalter crude effluent stream from the subsequent trial step as the initial value of the salt content. The above repetitive process is continued until the delta value of all streams are equal or less than the predetermined convergence tolerance value. The salt content of the desalter crude effluent stream from the final iteration is obtained as the salt content of the desalted crude oil.

The predetermined convergence tolerance value may be in a range of from about 0.1 to about 1 mg/l, or may be 1 mg/l.

In one or more embodiments, the measuring of the input parameters, obtaining target parameters such as target BS&W values, and iteratively calculating may be conducted at an interval in a range of greater than 0 second to about 60 seconds, such as in a range of from a lower limit selected from any one of 0.0001, 0.001, 0.01, and 0.1 seconds to an upper limit selected from any one of 0.1, 1, 10, 30 and 60 seconds, where any lower limit may be paired with any mathematically-compatible upper limit. The interval may be 1 second. Such interval may provide adequate data acquisition for continuous monitoring of the salt content.

The obtained salt content of the desalted crude oil may be shown, for example, using a display such as a computer monitor.

### A Method for Controlling a Salt Content of Desalted Crude Oil

In another aspect, embodiments disclosed herein relate to a method for controlling a salt content of desalted crude oil. The method includes providing a continuous crude oil desalting system, obtaining input parameters, and iteratively calculating a salt content of the desalter crude effluent stream according to salt mass balances. The method further includes comparing the salt content of the desalter crude effluent stream, such as the second desalter crude effluent stream, and a target salt content of the second desalter crude effluent stream, and adjusting at least one of the input parameters based on the iteratively calculated salt content of desalter crude effluent stream. The method is conducted to monitor and control the salt content of desalted crude oil without the use of a salt analyzer.

In one or more embodiments, the obtaining or measuring of input parameters (input process parameters, target parameters), and iteratively calculating the salt content included in the method for controlling a salt content of desalted crude oil may be any of the embodiments as previously described.

In one or more embodiments, the method includes comparing the salt content of desalted crude oil, such as the desalter crude effluent stream or second desalter crude effluent stream, and a target salt content of the desalted crude oil, such as the desalter crude effluent stream or the second desalter crude effluent stream. The target salt content may be lower than the maximum allowed salt content of the desalted crude oil based on a specification. For example, based on the maximum allowed salt content of the desalted crude oil of 10 lbs of salt per 1000 barrels of crude oil (PTB), the target salt content may be 9 PTB. The target salt content may be in a range of from a lower limit selected from any one of 6, 6.5 and 7 PTB to an upper limit selected from any one of 8, 8.5 and 9 PTB, where any lower limit may be paired with any upper limit.

When the salt content of the desalted crude oil as calculated reaches the target salt content value, the method further includes adjusting at least one input process parameter or target parameter, and repeating the iteratively calculating, comparing, and the adjusting until the calculated desalted crude oil value is lower than the target salt content value. The adjusting step may include adjusting at least one input process parameters, adjusting at least one target parameters, or combinations thereof. The adjusting may include adjusting of the flowrate of the wash water stream, adjusting of the salt and water contents of the wet crude feed stream, adjusting of the dehydrator or desalter process parameters such as mixing efficiency, adjusting of the percent recycling ratio of the water recirculation stream and combinations thereof. The adjustment of the salt and water contents of the wet crude feed stream may be conducted by modifying the blending of the raw crude oil in producing the wet crude feed stream in response to the changes to the water and salt content in the raw crude oil.

In one or more embodiments, the measuring of the input parameters, obtaining target parameters such as target BS&W values, iteratively calculating, and comparing the salt content of the desalter crude effluent stream the target salt content of the desalted crude oil may be conducted at an interval in a range of greater than 0 seconds to 60 seconds, such as in a range of from a lower limit selected from any one of 0.0001, 0.001, 0.01, and 0.1 seconds to an upper limit selected from any one of 0.1, 1, 10, 30 and 60 seconds, where any lower limit may be paired with any mathematically-compatible upper limit.

In one or more embodiments, the method includes providing an alert when the salt content of the desalted crude oil as calculated reaches the target salt content value. The alert may be provided visually via, for example, a notification on a computer display, illumination of a warning light, and/or audibly via, for example, a siren, or by any other notification method known in the art.

### EXAMPLES

The following examples are provided to illustrate embodiments of the present disclosure. The Examples are not intended to limit the scope of one or more embodiments of the present invention, and they should not be so interpreted.

### Example 1

An exemplary method for monitoring the salt content of the desalted crude oil was conducted in an existing desalting plant by using a continuous crude desalting system as provided in FIG. 1. Input parameters used in the present method are provided in Table 1.

TABLE 1

| Input parameters | Units | Value | Basis |
|---|---|---|---|
| Inlet Dry Crude (wet crude feed | MBD | 553 | From Existing flow |
| stream hydrocarbon content) | | | meter |
| Inlet Crude Water Out (wet crude | % | 11 | From Productions/ |
| feed stream water content) | | | analysis |
| Inlet Crude Salt Content (wet crude | mg/l | 143,300 | Lab analysis |
| feed stream salt content) | | | |
| Wash Water Stream Salt Content | mg/l | 242 | Lab analysis |
| Wash Water How Ratio | % | 1.5 | From Existing flow |
| | | | meter |
| Dehydrator Outlet BS&W | % | 0.2 | Specification |
| 1st Stage Desalter Outlet BS&W | % | 0.2 | Specification |
| 2nd Stage Desalter Outlet BS&W | % | 0.2 | Specification |
| Dehydrator Mixing Efficiency | % | 50 | Standard |
| | | | Requirement |
| 1st Stage Desalter Mixing | % | 50 | Standard |
| Efficiency | | | Requirement |
| 2nd Stage Desalter Mixing | % | 50 | Standard |
| Efficiency | | | Requirement |
| 1st Stage Recycle Percentage | % | 100 | PFD Configuration |
| 2nd Stage Recycle Percentage | % | 100 | PFD Configuration |

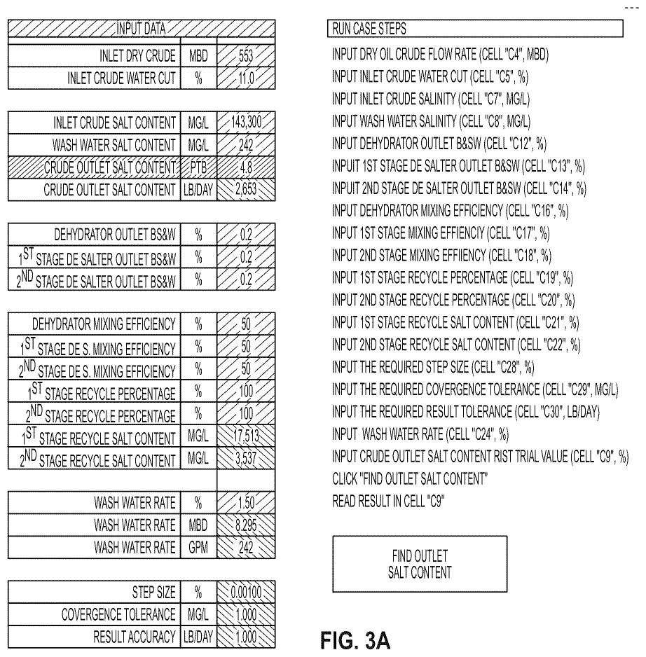

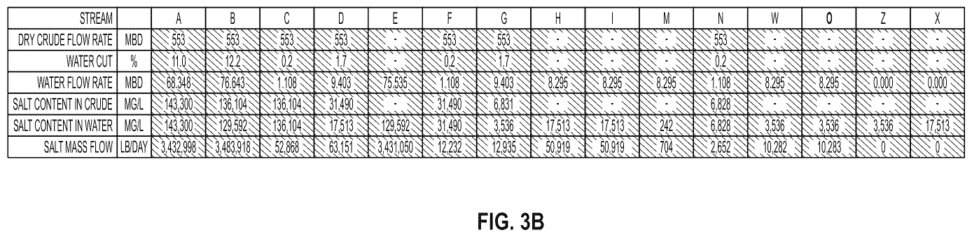

The iterative calculation based on the salt mass balances around the dehydrator, the first desalter and the second desalter was conducted using a spread sheet with Visual Basic codes with the input parameters as provided in Table 1. An exemplary user interface of the spread sheet showing the input parameters and calculated values, including the salt content of the desalted crude oil, is shown in FIGS. 3A-3B. The descriptions of the alphabetical labels corresponding to each stream in FIG. 3B are provided in Table 2. The “result accuracy” in FIG. 3A represents a convergence tolerance value of salt mass flow rate in lb/day.

TABLE 2

| Streams | Descriptions |
|---|---|
| A | Wet crude feed stream |
| B | Dehydrator feed stream |
| C | Dehydrator crude effluent stream |
| D | first stage desalter feed stream |
| E | Dehydrator water effluent stream |
| F | first stage desalter crude effluent stream |
| G | second stage desalter feed stream |
| H | first stage desalter water effluent stream |
| I | first water recirculcation stream |
| M | wash water stream |
| N | second stage desalter crude effluent stream |
| | (deslated crude oil) |
| O | second water recirculation stream |
| W | second stage desalter water effluent stream |
| X | first stage desalter water effluent stream |
| | (non-recirculated portion) |
| Z | second stage desalter water effluent stream |
| | (non-recirculated portion) |

The determined salt content of the desalted crude oil (second desalter crude effluent stream, stream N) was 4.8 pounds of salt per thousand barrels of crude oil (PTB).

The above exemplary method was conducted in an existing desalting plant once a day over a duration of a month. A sample of the desalted crude oil was obtained and analyzed for its salt content for comparison purposes. The calculated salt content based on the method of one or more embodiments, and the salt content obtained from the lab analysis are shown in Table 3. Table 3 also includes the mean, standard deviation and standard error of the calculated salt content and the salt content determined by the lab analysis for each day, and the average mean, standard deviation and standard error based on the salt contents calculated and obtained in the entire month.

TABLE 3

| | Salt Content (PTB) | | | | |
|---|---|---|---|---|---|
| | | Salt Balance | | | |
| | Lab | Approach | | Standard | Standard |
| Date | Reference | (Calculated) | Mean | Deviation | Error |
| Mar. 1, 2024 | 5.67 | 4.8 | 5.23 | 0.61 | 0.43 |
| Mar. 2, 2024 | 5.67 | 4.8 | 5.23 | 0.61 | 0.43 |
| Mar. 3, 2024 | 4.67 | 4.8 | 4.73 | 0.09 | 0.07 |
| Mar. 4, 2024 | 4.67 | 4.8 | 4.73 | 0.09 | 0.07 |
| Mar. 5, 2024 | 4.67 | 4.8 | 4.73 | 0.09 | 0.07 |
| Mar. 6, 2024 | 5.67 | 5.1 | 5.38 | 0.40 | 0.28 |
| Mar. 7, 2024 | 6.33 | 6 | 6.17 | 0.24 | 0.17 |
| Mar. 8, 2024 | 7.67 | 6.5 | 7.08 | 0.82 | 0.58 |
| Mar. 9, 2024 | 6.33 | 6 | 6.17 | 0.24 | 0.17 |
| Mar. 10, 2024 | 5.67 | 4.8 | 5.23 | 0.61 | 0.43 |
| Mar. 11, 2024 | 4.67 | 4.8 | 4.73 | 0.09 | 0.07 |
| Mar. 12, 2024 | 4.33 | 4.8 | 4.57 | 0.33 | 0.23 |
| Mar. 13, 2024 | 4.67 | 4.8 | 4.73 | 0.09 | 0.07 |
| Mar. 14, 2024 | 4.33 | 4.8 | 4.57 | 0.33 | 0.23 |
| Mar. 15, 2024 | 4.67 | 4.8 | 4.73 | 0.09 | 0.07 |
| Mar. 16, 2024 | 5.33 | 4.8 | 5.07 | 0.38 | 0.27 |
| Mar. 17, 2024 | 4.67 | 4.8 | 4.73 | 0.09 | 0.07 |
| Mar. 18, 2024 | 5.33 | 5.2 | 5.27 | 0.09 | 0.07 |
| Mar. 19, 2024 | 5.00 | 4.9 | 4.95 | 0.07 | 0.05 |
| Mar. 20, 2024 | 4.00 | 4.9 | 4.45 | 0.64 | 0.45 |
| Mar. 21, 2024 | 5.33 | 4.9 | 5.12 | 0.31 | 0.22 |
| Mar. 22, 2024 | 5.00 | 4.9 | 4.95 | 0.07 | 0.05 |
| Mar. 23, 2024 | 5.00 | 4.9 | 4.95 | 0.07 | 0.05 |
| Mar. 24, 2024 | 5.00 | 5 | 5.00 | 0.00 | 0.00 |
| Mar. 25, 2024 | 2.35 | 3.2 | 2.78 | 0.60 | 0.42 |
| Mar. 26, 2024 | 4.30 | 4.7 | 4.50 | 0.28 | 0.20 |
| Mar. 27, 2024 | 7.00 | 6.2 | 6.60 | 0.57 | 0.40 |
| Mar. 28, 2024 | 5.00 | 4.8 | 4.90 | 0.14 | 0.10 |
| Mar. 29, 2024 | 5.00 | 4.8 | 4.90 | 0.14 | 0.10 |
| Mar. 30, 2024 | 4.00 | 4.8 | 4.40 | 0.57 | 0.40 |
| Mar. 31, 2024 | 4.30 | 4.8 | 4.55 | 0.35 | 0.25 |
| Average | 4.88 | 4.97 | 4.92 | 0.30 | 0.21 |

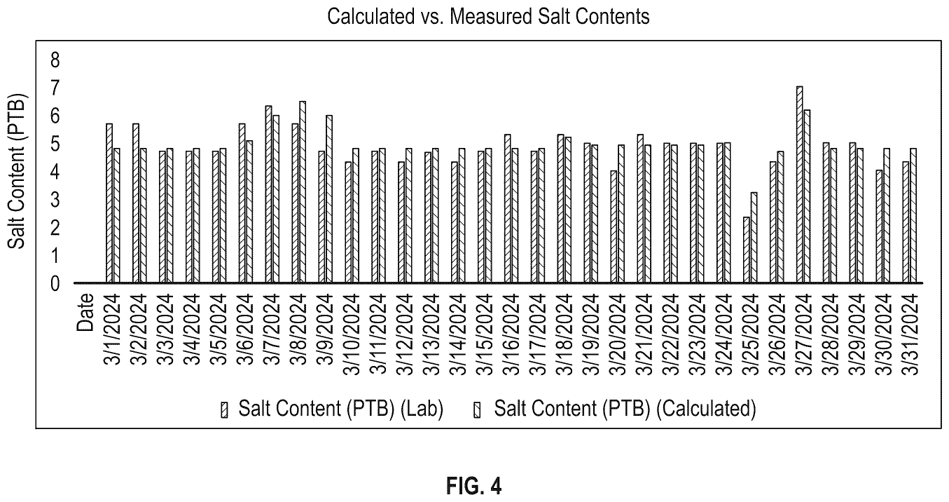

A graph of the calculated salt content and the salt content obtained via lab analysis as provided in Table 3 is shown in FIG. 4.

### BENEFITS

Embodiments of the present disclosure may allow the determination of the salt content of the desalted crude oil without the use of salt analyzers. Accordingly, embodiments of the present disclosure may provide various benefits.

The disclosed method allows continuous monitoring of the desalted crude oil salt content which enables proactive and robust process control and optimization. Because the salt content may be monitored continuously, it is possible to adjust process parameters such as wash water flow rates, desalter operation, and crude oil blending in response to the salt content deviation. Furthermore, prompt detection of anomalies and deviation from the target values is possible as a result of continuous monitoring. Such prompt response to the deviation and early detection of anomalies may result in optimal process efficiency and product quality.

Salt analyzers used in the art may be expensive to purchase, install, calibrate, and maintain. Removal of salt analyzers can eliminate or reduce capital costs and on-going operational expenses and tasks associated with the salt analyzers, such as maintenance, calibration, cleaning, replacement of consumables and operator training. One or more embodiments also eliminates the dependence on periodic sampling and lab analysis for salt content determination of the desalted crude oil.

Furthermore, removal of salt analyzers may result in reduced down time and improved accuracy and reliability in monitoring and controlling the effluent desalted crude oil quality. Salt analyzers can be prone to malfunctions, drift, or calibration errors, leading to downtime for troubleshooting and repairs. In addition, salt analyzers are sensitive instruments, and the results may be affected by various factors such as temperature variations, sample contamination, and chemical interference.

Elimination of salt analyzers may also streamline the desalting operation and increased flexibility in the desalting process design and operation, which may promote the process optimization and customization, and allow additional focus on other critical aspects of the desalting process. In addition, the calculation can be easily integrated into existing process control systems, allowing visualization of the salt content in addition to other parameters associated with the desalting process. The present method may also provide continuous insight into salt levels downstream of the wet crude handling unit, enabling operators to detect trends, anomalies, and deviations from target salt levels in real-time. This continuous monitoring capability allows for early detection of process upsets or deviations, facilitating prompt corrective actions to prevent adverse impacts on downstream processes or equipment.

Removal of the salt analyzers may also eliminate potential safety and environmental hazards associated with the analyzers. For example, hazards such as operator exposure to sour crude oil during the handling of the samples, or use of hazardous chemicals and agents required to operate the analyzers, may be eliminated, allowing enhanced safety and environment measures. Elimination of required samples and chemicals may also reduce the generated waste materials associated with the desalting process.

One or more embodiments of the present disclosure may lead to cost savings, simplified maintenance, improved enhanced safety, and environmental benefits, while maintaining accurate and effective salt content monitoring through methods without the use of salt analyzers.

Although only a few example embodiments have been described in detail above, those skilled in the art will readily appreciate that many modifications are possible in the example embodiments without materially departing from this invention. Accordingly, all such modifications are intended to be included within the scope of this disclosure as defined in the following claims.

What is claimed:

1. A method for monitoring a salt content of desalted crude oil, comprising:
feeding a dehydrator feed stream comprising a wet crude feed stream and a water recirculation stream to a dehydrator;
separating the dehydrator feed stream into a dehydrator crude effluent stream and a dehydrator water effluent stream;
feeding a desalter feed stream comprising the dehydrator crude effluent stream and a wash water stream to a desalter unit;
mixing the desalter feed stream in the desalter unit;
separating the desalter feed stream into a desalter crude effluent stream and a desalter water effluent stream;
recirculating at least a portion of the desalter water effluent stream to the dehydrator as the water recirculation stream;
measuring:
a flowrate of:
the wet crude feed stream,
the wash water stream, and
the water recirculation stream,
a water content of the wet crude feed stream, and
a salt content of the wet crude feed stream and the wash water stream;
obtaining a target basic sediment and water (BS&W) of the dehydrator crude effluent stream and the desalter crude effluent stream; and
iteratively calculating a salt content of the desalter crude effluent stream according to salt mass balances around the dehydrator and the desalter, based on input parameters and a trial salt content value of the desalter crude effluent stream,
wherein the input parameters comprise:
the flowrate of:
the wet crude feed stream,
the wash water stream, and
the water recirculation stream;
the water content of the wet crude feed stream;
the salt content of the wet crude feed stream and the wash water stream; and
the target BS&W of the dehydrator crude effluent stream and the desalter crude effluent stream.

2. The method according to claim 1, wherein the iterative calculation of the salt content of the desalter crude effluent stream comprises:
an initial trial step of:
selecting an initial trial salt content value of the desalter crude effluent stream; and
determining an initial trial salt content of:
the dehydrator crude effluent stream,
the dehydrator water effluent stream, and
the desalter water effluent stream,
according to the salt mass balances based on the input parameters and the initial trial salt content value of the desalter crude effluent stream;
a subsequent trial step of:
selecting a subsequent trial salt content value of the desalter crude effluent stream, wherein the subsequent trial salt content value differs from the initial trial salt content value by a predetermined step amount; and
determining a subsequent trial salt content of:
the dehydrator crude effluent stream,
the dehydrator water effluent stream, and
the desalter water effluent stream,
according to the salt mass balances based on the input parameters and the subsequent trial salt content value of the desalter crude effluent stream;
a comparing step of:
obtaining a delta value, which is a difference between the initial trial salt content and the subsequent trial salt content of each of the dehydrator crude effluent stream, the dehydrator water effluent stream, and the desalter water effluent stream; and
repeating the initial trial step, the subsequent trial step and the comparing step by substituting the subsequent trial salt content as the initial trial salt content until all of the delta value is equal or less than a predetermined convergence tolerance value.

3. The method of claim 2, wherein the predetermined step amount is in a range of greater than 0% to 0.2% of the initial trial salt content value of the desalter crude effluent stream.

4. The method of claim 2, wherein the predetermined convergence tolerance value is in a range of from 0.1 mg/l to 1 mg/l.

5. The method of claim 1, wherein the input parameters further comprise a mixing efficiency of the dehydrator, and a mixing efficiency of the desalter.

6. The method of claim 1, wherein the measuring, obtaining a target basic sediment and water value, and the iteratively calculating are conducted at an interval in a range of greater than 0 second and 60 seconds or less.

7. The method of claim 1, wherein the salt content of the desalted crude oil is determined without a salt analyzer.

8. A method for controlling a salt content of desalted crude oil, comprising:
feeding a dehydrator feed stream comprising a wet crude stream and a first water recirculation stream to a dehydrator;
separating the dehydrator feed stream into a dehydrator crude effluent stream and a dehydrator water effluent stream;
feeding a first desalter feed stream comprising the dehydrator crude effluent stream and a second water recirculation stream to a first desalter unit;
mixing the first desalter feed stream in the first desalter unit;
separating the first desalter feed stream into a first desalter crude effluent stream and a first desalter water effluent stream;
recirculating at least a portion of the first desalter water effluent stream to the dehydrator as the first water recirculation stream;
feeding a second desalter feed stream comprising the first desalter crude effluent stream and a wash water stream to a second desalter unit;
mixing the second desalter feed stream in the second desalter unit;
separating the second desalter feed stream into a second desalter crude effluent stream and a second desalter water effluent stream;
recirculating at least a portion of the second desalter water effluent stream to the first desalter unit as the second water recirculation stream;
periodically measuring:
a flowrate of the wet crude feed stream, the wash water stream, the first water recirculation stream, and the second water recirculation stream;
a water content of the wet crude feed stream; and
a salt content of the wet crude feed stream and the wash water stream;
obtaining target parameters comprising a target basic sediment and water (BS&W) of the dehydrator crude effluent stream, the first desalter crude effluent stream and the second desalter crude effluent stream;
iteratively calculating a salt content of the second desalter crude effluent stream according to salt mass balances around the dehydrator, the first desalter and the second desalter, based on input process parameters, the target parameters and a trial salt content value of the second desalter crude effluent stream;
comparing the salt content of the second desalter crude effluent stream and a target salt content of the second desalter crude effluent stream; and
when the salt content of the second desalter crude effluent is equal or greater than the target salt content of the second desalter crude effluent stream:
adjusting at least one of the input process parameter or the target parameter; and
repeating the iteratively calculating, the comparing, and the adjusting until the salt content of the second desalter crude effluent stream is less than the target salt content,
wherein the input process parameters comprise:
the flowrate of:
the wet crude feed stream,
the wash water stream,
the first water recirculation stream, and
the second water recirculation stream;
the water content of the wet crude feed stream; and
the salt content of the wet crude feed stream and the wash water stream.

9. The method according to claim 8, wherein the iterative calculation of the salt content of the desalter crude effluent stream comprises:
an initial trial step of:
selecting an initial trial salt content value of the desalter crude effluent stream; and
determining an initial trial salt content of:
the dehydrator crude effluent stream,
the dehydrator water effluent stream,
the first crude effluent stream,
the first desalter water effluent stream,
the second desalter crude effluent stream, and
the second desalter water effluent stream,
according to the salt mass balances based on the input process parameters, the target parameters and the initial trial salt content value of the desalter crude effluent stream;
a subsequent trial step of:
selecting a subsequent trial salt content value of the desalter crude effluent stream, wherein the subsequent trial salt content value differs from the initial trial salt content value by a predetermined step amount; and
determining a subsequent trial salt content of:
the dehydrator crude effluent stream,
the dehydrator water effluent stream,
the first desalter crude effluent stream,
the first desalter water effluent stream,
the second desalter crude effluent stream, and
the second desalter water effluent stream,
according to the salt mass balances based on the input process parameters, the target parameters and the subsequent trial salt content value of the second desalter crude effluent stream;
a comparing step of:
obtaining a delta value, which is a difference between the initial trial salt content and the subsequent trial salt content, for each of the dehydrator crude effluent stream, the dehydrator water effluent stream, and the first desalter crude effluent stream, the first desalter water effluent stream, and the second desalter water effluent stream; and
repeating the initial trial step, the subsequent trial step and the comparing step by substituting the subsequent trial salt content as the initial trial salt content until all of the delta value is equal or less than a predetermined convergence tolerance value.

10. The method of claim 9, wherein the predetermined step amount is in a range of greater than 0% and 0.2% or less of the initial trial salt content value of the desalter crude effluent stream.

11. The method of claim 9, wherein the predetermined convergence tolerance value is in a range of from 0.1 mg/l to 1 mg/l.

12. The method of claim 8, wherein the target parameters further comprise a percent recycling ratio of the first water recirculation stream and the second water recirculation stream.

13. The method of claim 8, wherein the target parameters further comprise a mixing efficiency of the dehydrator, the first desalter and the second desalter.

14. The method of claim 8, wherein the periodically measuring, the iteratively calculating, the obtaining target parameters, and comparing the salt content of the second desalter crude effluent stream and a target salt content of the second desalter effluent stream are conducted at an interval in a range of greater than 0 seconds and 60 seconds or less.

15. The method of claim 8, wherein the salt content of the desalted crude oil is determined without a salt analyzer.
